(19) United States
(12) Patent Application Publication
(10) Pub. No.: US 2007/0288076 A1
(43) Pub. Date: Dec. 13, 2007

(54) BIOLOGICAL TISSUE STIMULATOR WITH FLEXIBLE ELECTRODE CARRIER

(76) Inventors: Cherik Bulkes, Sussex, WI (US); Stephen Denker, Mequon, WI (US)

Correspondence Address: QUARLES & BRADY LLP, 411 E. WISCONSIN AVENUE, SUITE 2040, MILWAUKEE, WI, 53202-4497, US

(21) Appl. No.: 11/759,476

(22) Filed: Jun. 7, 2007

Related U.S. Application Data
(60) Provisional application No. 60/811,501, filed on Jun. 7, 2006.

(51) Int. Cl. A61N 1/05

(52) U.S. Cl. 607/116

(57) ABSTRACT

A biological tissue stimulating apparatus is provided that is adapted for intraluminal implantation is an animal. The apparatus includes a flexible electrode carrier on which a plurality of exposed electrodes formed on a flexible insulating layer wherein the electrodes are to contact the tissue being stimulated. A separate electrical conductor extends from each electrode to a control circuit. The control circuit programmably selects pairs of electrodes for transluminally stimulating the biological tissue. The flexible electrode carrier is adapted to be deployed in a lumen of an organ of the animal, for example a blood vessel, in a spirally coiled form that expands upon being properly located in the lumen to secure the flexible electrode carrier against on to the inner wall of the lumen.

### CROSS-REFERENCE TO RELATED APPLICATIONS

This application claims benefit of U.S. Provisional Patent Application No. 60/811,501 filed on Jun. 7, 2006.

### STATEMENT REGARDING FEDERALLY SPONSORED RESEARCH OR DEVELOPMENT

Not Applicable

### BACKGROUND OF THE INVENTION

1. Field of the Invention

The present invention relates to implantable medical devices, which deliver energy to stimulate tissue for the purposes of providing therapy to the tissue of an animal, and in particular to a stimulator with flexible electrode carrier capable of conforming to variable diameters and lengths for implantation.

2. Description of the Related Art

A remedy for a patient with one of several physiological ailments is to implant an electrical stimulation device. An electrical stimulation device is a small electronic apparatus that stimulates an organ, nerves leading to that organ or part of an organ. It includes a stimulation pulse generator, implanted in the patient, which produces electrical pulses to stimulate the organ or to change its metabolism or function. Electrical leads extend from the pulse generator to electrodes placed adjacent to specific regions of the organ, which when electrically stimulated provide therapy to the patient.

An improved apparatus for physiological stimulation of a tissue includes a radio frequency (RF) receiver implanted as part of a transvascular platform that comprises an electronic capsule containing stimulation circuitry connected to at least one electrode assembly. The electrode assembly has a carrier on which one or more electrodes are mounted. The stimulation circuitry receives the radio frequency signal and from the energy of that signal derives an electrical voltage. The electrical voltage is applied by the stimulation circuitry in the form of suitable waveforms to the electrodes, thereby stimulating the tissue.

In addition to making proper electrode to tissue contact, it is important that an electrode assembly be flexible in terms of the ratio of the expanded state diameter to the collapsed state diameter. Therefore, it is desirable that the electrode carrier have a degree of flexibility. This allows the device to fit in a variety of locations, even tapering blood vessels, without occluding the vasculature while at the same time provide error-free contacts for expected stimulation as part of the stimulation apparatus.

### SUMMARY OF THE INVENTION

An apparatus is disclosed for stimulating biological tissue adapted for intraluminal implantation using a flexible electrode carrier. The flexible electrode carrier includes a plurality of electrodes formed on a flexible insulating layer, wherein the electrodes are exposed in order to contact the tissue to be stimulated. A separate electrical conductor connects each electrode to a control circuit that programmably selects different combinations of the electrodes for transluminally stimulating the biological tissue. The flexible electrode carrier is adapted to be deployed in a lumen, for example a blood vessel. The flexible electrode carrier initially is in a diametrically contracted, coiled state that enables insertion into the lumen and then when properly located, is expanded against the inner wall of the lumen to secure the carrier in place.

The programmable selection of electrodes for stimulation is dynamically chosen and allows polarity reversal. The stimulation may be unipolar, bipolar or multi-polar. The order of the electrode selection for stimulation may be a predefined temporal sequence. A number of exposed electrodes may be selected to stimulate at least one site or multiple sites in the lumen. The inventive aspect also allows for different stimulation protocols are chosen to stimulate different multiple sites in the lumen. The stimulation site may be dynamically determined by sensing responses from multiple sites and selecting the most responsive site.

### BRIEF DESCRIPTION OF DRAWINGS

FIG. 1 schematically depicts external and internal subsystems of a wireless transvascular platform for animal tissue stimulation;

FIG. 2A illustrates an electrode carrier of the internal subsystem in an unfolded and uncoiled state;

FIG. 2B illustrates the electrode carrier folded longitudinally;

FIG. 2C illustrates an electrode carrier wound in a spiral;

FIG. 3 is a longitudinal cross section through a portion of the electrode carrier;

FIGS. 4A and B respectively show the electrode carrier deployed in a uniform cylindrical blood vessel and in a tapering blood vessel; and

FIG. 5 is a schematic diagram of the electrode carrier connected to implanted electrical circuitry that applies a stimulation signal to the electrode carrier.

### DETAILED DESCRIPTION OF THE INVENTION

Although the present invention is being described in the context of an intravascular stimulator and although the present electrode carrier is particularly adapted for implantation in a lumen of an organ of an animal, the inventive concepts can be utilized in devices for stimulating other organs and in devices implanted elsewhere in the body.

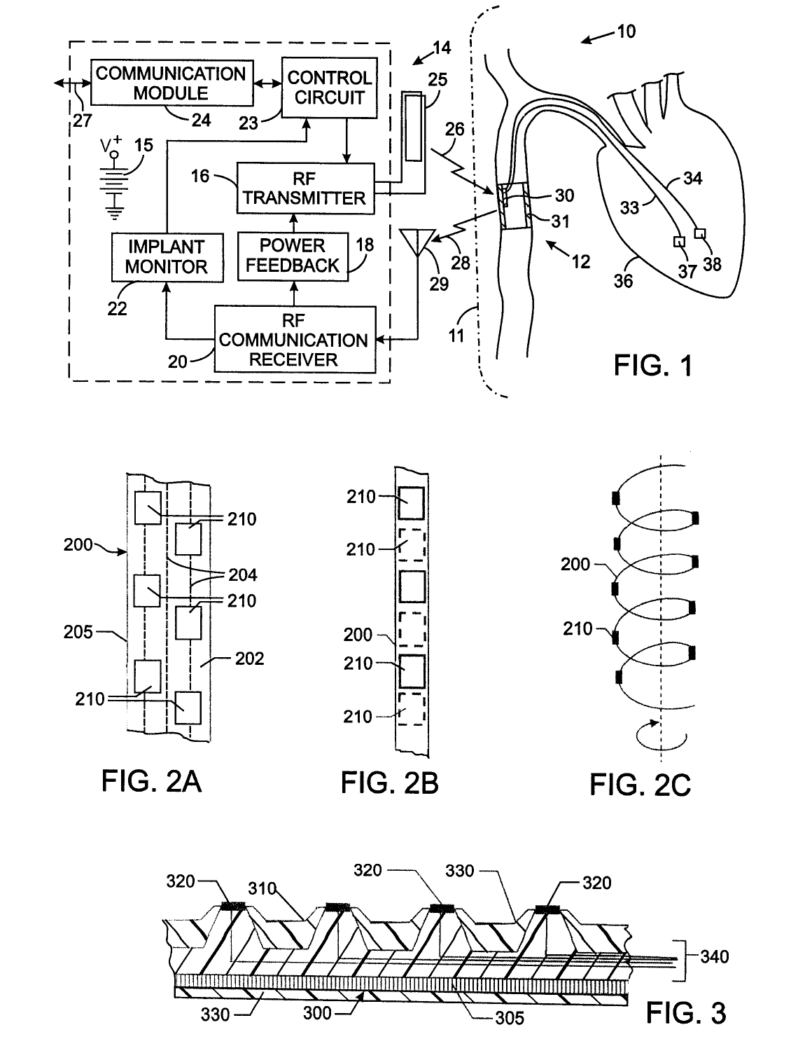

With initial reference to FIG. 1, a transvascular platform 10 for tissue stimulation includes an extracorporeal power source 14 and a stimulator 12 implanted inside the body 11 of an animal. The extracorporeal power source 14 communicates with the implanted stimulator 12 via wireless signals. The extracorporeal power source 14 includes a rechargeable battery 15 that powers a transmitter 16 which sends a first radio frequency (RF) signal 26 via a first transmit antenna 25 to the stimulator 12. The first RF signal 26 provides electrical power to the stimulator 12. The transmitter 16 pulse width modulates the first RF signal 26 to control the amount of power being supplied. The first radio frequency signal 26 also carries control commands and data to configure the operation of the stimulator 12.

The implanted stimulator 12 includes the electronic circuit 30 that is mounted on an circuit carrier 31 and includes an radio frequency transceiver and a tissue stimulation circuit similar to that used in previous pacemakers and defibrillators. That circuit carrier 31 is positioned in a large blood vessel 32, such as the inferior vena cava (IVC), for example. One or more, electrically insulated electrical cables 33 and 34 extend from the electronic circuit 30 through the coronary blood vessels to locations in the heart 36 where pacing and sensing are desired. The electrical cables 33 and 34 terminate at stimulation electrodes located on electrode assemblies 37 and 38 at those locations. Each electrode assemblies 37 and 38 has a plurality of contact electrodes.

The present invention provides means to dynamically select different combinations of the contact electrodes for stimulation purposes. FIG. 5 schematically shows a preferred means by which this is accomplished. The electronic circuit 30 of the implanted stimulator 12 has a first receive antenna 40 tuned to pick-up a first RF signal 26 from the extracorporeal power source 14. The signal from the first receive antenna 40 is applied to a discriminator 42 that separates the received signal into power and data components. Specifically, a rectifier 44 functions as a power circuit which extracts energy from the first RF signal to produce a DC voltage (VDC) that is applied across a storage capacitor 48 from which electrical power is supplied to the other components of the stimulator 12. The DC voltage is monitored by a voltage feedback detector 50 that provides an indication of the capacitor voltage level to a data transmitter 52 which sends that indication from a second transmit antenna 54 via the second radio frequency signal 28 to the extracorporeal power source 14.

Commands and control data carried by the first RF signal 26 are extracted by a data detector 46 in the stimulator 12 and fed to an analog, digital or hybrid controller 56. That controller 56 receives physiological signals from sensors 55 implanted in the animal. In response to the sensor signals, the controller 56 activates a stimulation circuit 57 that comprises a stimulation signal generator 58 which applies a stimulation voltage via selection logic 60 to the electrode assemblies 37 and 38 (only assembly 37 is illustrated), thereby stimulating the adjacent tissue in the animal.

Referring again to FIG. 1, the extracorporeal power source 14 receives the second radio frequency signal 28 carrying data sent by the stimulator 12. That data include the supply voltage level as well as physiological conditions of the animal, status of the stimulator and trending logs, that have been collected by the implanted electronic circuit 30, for example. To receive that second RF signal 28, the extracorporeal power source 14 has a radio frequency communication receiver 20 connected to a second receive antenna 29. A power feedback module 18 extracts data regarding the supply voltage level in the stimulator 12 to control the generation of the first RF signal 26 accordingly. An implant monitor 22 extracts stimulator operational data from the second RF signal 28, which data are sent to a control circuit 23. An optional communication module 24 may be provided to exchange data and commands via a communication link 27 with other external apparatus (not shown), such as a programming computer or patient monitor so that medical personnel can review the data or be alerted when a particular condition exists. The communication link 25 may be a wireless link such as a radio frequency signal or a cellular telephone connection.

Focusing on an intravascular stimulation system, each electrode assembly 37 or 38 has an electrode carrier that provides a stable anchor for the electrodes, such that positional stability is ensured. Thus the electrode carrier has to provide sufficient tension to adhere to the blood vessel wall to prevent inadvertent dislodgement. The electrode carrier also has to be collapsible to enable insertion via a small catheter in a manner that minimizes the insult to the patient. The electrode carrier can be delivered in a radially constrained configuration, e.g. by placing the electrodes within a delivery sheath or tube and retracting the sheath at the target site. After being properly located, each electrode carrier 37 and 38 a restraint that maintains the collapsed state is released to allow the electrode carrier to self-expand. In that expanded state, the electrode carrier retains sufficient flexibility so as not to interfere with the natural motility of the containing vessel lumen. A shape memory material, such as Nitinol or stainless steel, can be deployed in the lead and electrode structure to provide this ability.

A section of an electrode carrier 200 is shown in FIG. 2A as an unfolded and unrolled ribbon formed by a layer 205 of a biocompatible, electrical insulation material, such as urethane or silicone, with a plurality of stimulation contact electrodes 210 mounted on one major surface 202. A biocompatible material is a substance that is capable of being used in the human body without eliciting a rejection response from the surrounding body tissues, such as inflammation, infection, or an adverse immunological response. The contact electrodes 210 are made of biocompatible, electrically conductive material, such as gold, stainless steel or carbon. The electrode carrier 200 is folded lengthwise as shown in FIG. 2B so that the major surface 202 forms opposite front and back surfaces of the resultant object. Some of the contact electrodes 210 are located on each of those opposite surfaces with solid squares depicting contact electrodes 210 in the front surface and the dotted squares represent the contact electrodes at back surface of the folded carrier. Additionally, the electrode carrier 200 can be wound in a spiral coil as shown in FIG. 2C. For certain applications, it may be advantageous to embed wires 204 of a shape memory material (see FIG. 2A) to reinforce the insulation layer 205 so that the electrode carrier attains a coiled shape upon release inside the lumen of the animal's organ.

Another aspect of the electrode carrier design is to maintain end portions to be substantially less stiff than the intermediate portion to reduce tissue trauma. The main intermediate portion may include a ladder-like structure having edge elements separated by connector elements. The end portions may have inwardly-tapering portions with blunt tips. The inwardly tapering portions may have lengths greater than their widths. The intermediate portion also may be designed to have longitudinal sections with different radial stiffnesses.

Referring to FIG. 3, the ribbon electrode carrier 300 has an optional substrate 305 that provides structure or shape memory and which preferably is made of a shape memory material, such as Nitinol or stainless steel. The contact electrodes 320 are mounted on a surface of an insulation layer 310 of electrically insulating material, such as urethane or silicone, that is attached to and reinforced by the substrate 305. The contact electrodes 320 are made up of biocompatible conductive material and are connected to control electronics through the conductors, such as wires 340 that are encased in the insulation layer 310. These electrical conductors are preferably formed by a fatigue resistant material, such as stainless steel, Nitinol or MP35N nickel-cobalt based alloy. MP35N is a trademark of SPS Technologies, Inc. The entire electrode assembly, except for the contact electrodes 320, is covered with a biocompatible insulation layer 330 such as urethane.

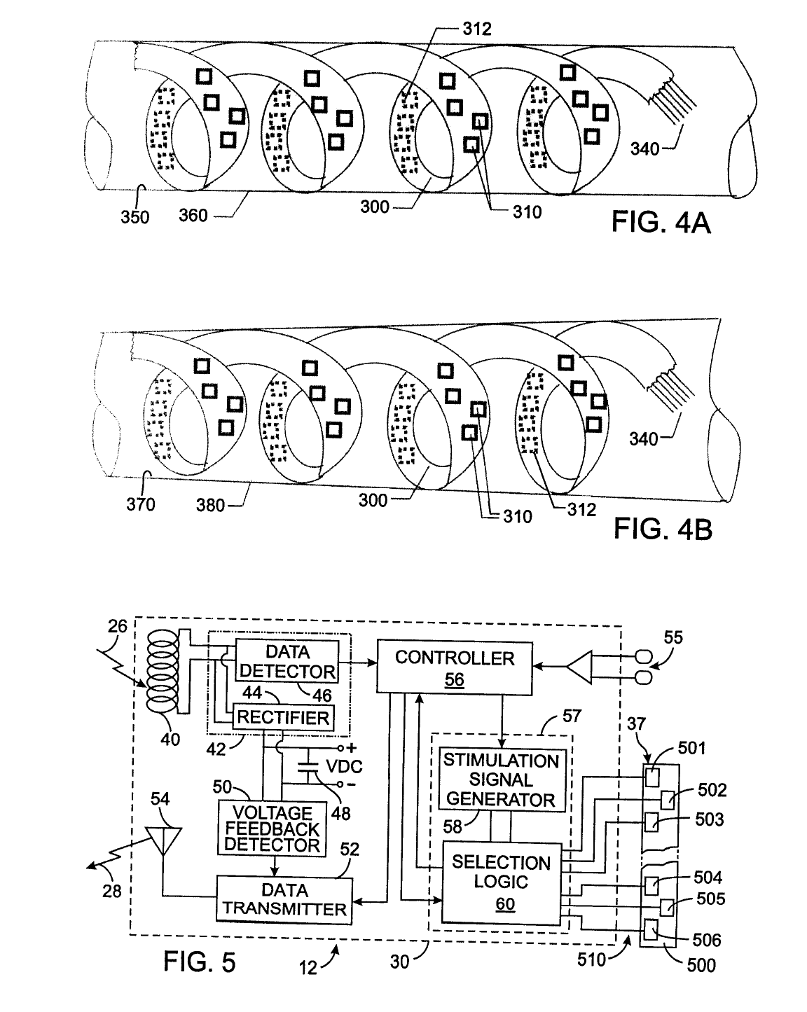

FIG. 4A is a rendering of the flexible ribbon electrode carrier 300 in a wound in a spiral and implanted in the lumen 350 of a cylindrical blood vessel 360 of an animal. The conductors 340 are illustratively represented as tracking along the length of the ribbon although alternative combinations such as along the side are possible. These conductors are electrically insulated from one another. FIG. 4B is a three-dimensional schematic rendering of the spiral wound, ribbon electrode carrier 300 in a coiled form located in the lumen 370 of a tapered blood vessel 380. In both types of blood vessels, the length of the ribbon electrode carrier 300 may be variable to suit the application. Note that the configuration is flexible to adapt to any size of the vessel diameter including variable diameter of the vessel. Furthermore, the coiled shape does not occlude any branches extending from the main blood vessel.

The present invention provides means to dynamically select certain ones of the contact electrodes for stimulation purposes. FIG. 5 schematically shows how this could be accomplished. The contact electrodes 501-506 on electrode carrier 500 are connected by conductors 510 to a selection logic 60 that is being programmably controlled by controller 56. For example, the controller 56 monitors each contact electrode 501-506 and selects the two contact electrodes that can provide optimal stimulation. The controller 56 also senses anatomical electrical signals at the electrode sites and responds by choosing appropriate sites for optimizing stimulation. In one case, contact electrodes 501 and 502 are optimal and are chosen through the selection logic 60 for stimulating the tissue. Here the stimulation voltage waveform produces by the stimulation signal generator 58 is routed by the selection logic 60 to those selected contact electrodes 501 and 502. The polarity of these contact electrodes chosen by the selection logic 60 as well. In one instance, electrode 501 is the positive contact electrode and electrode 502 is the negative counterpart. In another instance, the polarity contact electrodes 501 and 502 is reversed. It should be noted that unipolar, bipolar and multi-polar electrical stimulation can be employed. At other times, other pair combinations of contact electrodes, e.g. contact electrodes 503 and 506, are chosen based on their proximity to the desired stimulation site.

In some embodiments contemplated in the present invention, multiple contact electrodes 501-506 can be sequentially activated for stimulating tissue in a progressive manner. This sequencing can be used to perform muscle or neuronal activation. As an example, the stimulation voltage is applied to contact electrodes 501 and 506 for a preset time, followed by contact electrodes 502 and 505, then contact electrodes 503 and 504. This sequence can be repeated for a desired amount of time or a desired number of times.

It should be noted that different stimulation protocols can be employed with the multiple electrodes available for selection. Each stimulation protocol includes specifying waveforms for stimulation, duty cycles, durations, amplitudes, shapes of waveforms, and spatial and temporal sequences of waveforms. The protocols are programmably selected by the control circuit and commands are issued to the stimulation circuitry including multiple electrodes formed on the flexible electrode carrier in a deployed state in the lumen. The multi-electrode configuration also allows for different types of stimulation to be carried out concurrently or in an alternating fashion.

In one embodiment, contact electrodes on the flexible carrier may be adapted to stimulate a single site with multiple electrodes. In another embodiment, contact electrodes on the flexible carrier may be adapted to stimulate multiple sites with multiple electrodes. In yet another embodiment, stimulation sequence and/or duration in multiple distributed electrodes may be spatially and/or temporally varied. In yet another embodiment, stimulation site may be dynamically determined adaptively by sensing responses from multiple sites and selecting the most responsive site. This kind of dynamic determination may be repeated after certain amount of time.

In some embodiments of the current invention, sensed outputs of all the applicable electrodes may be analyzed before choosing the signals from best electrodes.

In some embodiments, electrode sites making the best contact may be chosen for stimulation.

For deployment, the spiral coiled electrode carrier, is wound about a catheter shaft in torqued compression by securing the ends of the coil on a catheter shaft. The ends are released by, for example, pulling on release wires once at the target site in the animal. Alternatively, the electrode carrier can be maintained in its reduced-diameter condition by a sleeve that is retracted to release the flexible electrode carrier. In a third approach, a balloon is used to expand the electrode carrier at the target site. The electrode carrier typically extends past its elastic limit so that it remains in its expanded state after the balloon is deflated.

Various modifications of the flexible electrode carrier can be used for tissue stimulation of different organs of an animal. In fact, the device can be scaled appropriately to be applicable to be placed in any lumen for stimulation purposes and not just limited to the vascular system. Therefore, the scope of the electrode configurations and flexible electrode carrier assembly should be viewed to encompass all such endoluminal prosthetic alternatives as elucidated in the ensuing claims.

1. An apparatus for stimulating biological tissue and adapted for implantation in a lumen of an organ of an animal, said apparatus comprising:
an electrode assembly having a flexible electrode carrier that includes a flexible layer of electrical insulating material with a major surface and a plurality of electrodes formed on the major surface of the electrode carrier for contacting the biological tissue upon implantation into the animal, the electrode carrier coiled into a spiral that is diametrically contractable for insertion into the animal and expandable to secure the electrode assembly in the lumen;
a plurality of electrical conductors each being connected to one of the plurality of electrodes; and
a stimulation circuit connected to the plurality of electrical conductors for generating a stimulation voltage and selecting a pair of the plurality of electrodes to which the stimulation voltage is applied stimulate the biological tissue.

2. The apparatus as recited in claim 1 wherein the stimulation circuit dynamically selects a pair of the plurality of electrodes.

3. The apparatus as recited in claim 1 wherein the stimulation circuit varies a polarity of the stimulating voltage applied to the pair of the plurality of electrodes.

4. The apparatus as recited in claim 1 wherein the stimulation circuit applies a unipolar stimulating voltage to the pair of the plurality of electrodes.

5. The apparatus as recited in claim 1 wherein the stimulation circuit applies a bipolar stimulating voltage to the pair of the plurality of electrodes.

6. The apparatus as recited in claim 1 wherein the stimulation circuit applies a multi-polar stimulating voltage to the pair of the plurality of electrodes.

7. The apparatus as recited in claim 1 wherein the stimulation circuit applies the stimulating voltage to different pairs of the plurality of electrodes in a predefined temporal sequence.

8. The apparatus as recited in claim 1 wherein the flexible layer contains a shape memory material.

9. The apparatus as recited in claim 1 wherein the flexible electrode carrier further comprises a substrate of a shape memory material attached to the flexible layer.

10. The apparatus as recited in claim 1 wherein the flexible layer is folded lengthwise.

11. The apparatus as recited in claim 1 wherein the flexible electrode carrier further comprises a biocompatible exterior layer encasing all components of the electrode assembly except the plurality of electrodes.

12. The apparatus as recited in claim 1 wherein pair of the plurality of electrodes is chosen to stimulate at least one site in the lumen.

13. The apparatus as recited in claim 1 wherein a plurality of stimulation protocols is selected to stimulate at least one site in the lumen.

14. The apparatus as recited in claim 1 wherein different stimulation protocols are chosen to stimulate multiple sites in the lumen.

15. The apparatus as recited in claim 1 wherein a plurality of exposed electrodes is selected to stimulate multiple sites in the lumen.

16. The apparatus as recited in claim 1 wherein a stimulation site is dynamically selected by sensing responses from multiple sites in the lumen and selecting one of the multiple sites that best satisfies a predetermined criteria.

17. The apparatus as recited in claim 1 wherein the electrode assembly is deployed in the lumen of a blood vessel.

18. The apparatus as recited in claim 17 wherein the flexible electrode carrier conforms to the blood vessel that has a diameter that varies.

19. An apparatus for stimulating biological tissue and adapted for intravascular implantation in an animal, said apparatus comprising:
a control circuit;
an electrode assembly having a flexible layer of electrical insulating material with a major surface, a plurality of electrodes formed on the major surface for contacting the biological tissue upon implantation into the animal, the flexible layer coiled into a spiral that is diametrically contractable for insertion into the animal and expandable for securing the electrode carrier in the vasculature of the animal;
a plurality of electrical conductors each being connected to one of the plurality of electrodes; and
a stimulation circuit connected to the plurality of electrical conductors and to the control circuit for generating and applying a stimulating voltage to a selected pair of the plurality of electrodes to stimulate transvascularly the biological tissue.

20. The apparatus as recited in claim 19 wherein the stimulation circuit dynamically selects a pair of the plurality of electrodes.

21. The apparatus as recited in claim 19 wherein at least part of each of the plurality of electrical conductors is embedded inside the flexible layer of electrical insulating material.
